(19) United States
(12) Patent Application Publication
(10) Pub. No.: US 2021/0395830 A1
(43) Pub. Date: Dec. 23, 2021

(54) USES OF METHYLATED LOCI IN SONIC HEDGEHOG MEDULLOBLASTOMA

(71) Applicants: Guangzhou Kingmed Center for Clinical Laboratory, Guangzhou City (CN); Guangzhou KingMed Diagnostics Group Co., Ltd., Guangzhou, Guangdong (CN); Guangzhou KingMed Transformative Medicine Institute Co. Ltd., Guangzhou, Guangdong (CN)

(72) Inventors: Jingxing LIU, Guangzhou (CN); Weiwei ZHAO, Guangzhou (CN); Shihui YU, Guangzhou (CN); Mingming SUN, Guangzhou (CN); Baixue CHEN, Guangzhou (CN); Changming HU, Guangzhou (CN); Xin BI, Guangzhou (CN); Yishe WANG, Guangzhou (CN)

(21) Appl. No.: 17/289,803

(22) PCT Filed: Dec. 27, 2019
(86) PCT No.: PCT/CN2019/129127

§ 371 Date: Apr. 29, 2021

(30) Foreign Application Priority Data
Feb. 1, 2019 (CN) 201910105563.X

(51) Int. Cl. C12Q 1/6886

(52) U.S. Cl. CPC C12Q 1/6886; C12Q 2600/154

(57) ABSTRACT

Provided is a use of methylated locus in Sonic Hedgehog subgroup medulloblastoma, which relates to the technical field of tumor classification and detection. Specifically, the present disclosure provides a use of a reagent for detecting a methylation level of at least one genetic locus selected from ROBO4, OTX2OS1, AHRR, DKK4, or PART1 in a biological sample in the preparation of a diagnostic reagent or a diagnostic kit for Sonic Hedgehog subgroup medulloblastoma. Based on analysis and comparison of data from a large number of Sonic Hedgehog subgroup medulloblastoma samples and other medulloblastoma subgroups samples the methylation level of a genetic locus in the above genes can be used as a referenced indicator for the diagnosis of Sonic Hedgehog subgroup medulloblastoma. Therefore, only a few methylated loci are targeted and detected, and MB SHE can be classified and identified, thereby reducing diagnostic difficulty and cost of Sonic Hedgehog subgroup medulloblastoma.

### CROSS-REFERENCE TO RELATED APPLICATIONS

This application claims priority to PCT Application No. PCT/CN2019/129127, having a filing date of Dec. 27, 2019, which is based on Chinese Application No. 201910105563.X, having a filing date of Feb. 1, 2019, the entire contents both of which are hereby incorporated by reference.

### FIELD OF TECHNOLOGY

The following relates to the technical field of tumor classification and detection, particularly, to a use of a methylated locus in Sonic Hedgehog medulloblastoma (MBSHH).

### BACKGROUND

Medulloblastoma is a most common malignant brain tumor in children. Approximately 85% of medulloblastoma occurs in children under 18 years old, and one child in every five children with brain tumor suffers from medulloblastoma. In recent years, the survival rate of patients with medulloblastoma has been increased, but the motality is still high. Even though the patients have been treated successfully, they often suffer from sequelae in nervous and endocrine systems.

The incidence of medulloblastoma is about 0.71 per 100,000 people. In the United States, there are 400 to 500 children with such disease every year. The peak period of this disease incidence occurs in children aged 3 to 6 years, but the disease is very rare in people beyond the age of 50. There is no report that the incidence of medulloblastoma is affected by the biochemical environment. However, certain genetic diseases may lead to the occurrence of medulloblastoma, and about 7% of patients may have genetic mutations in germ cells. A few patients suffer medulloblastoma due to familial heredity.

At present, it has been recognized worldwide that medulloblastoma is not a single disease, but a brain tumor consisting of many different molecular subgroups. Each subgroup has significant differences in genetic, demographic, and clinical characteristics. In the most recent mainstream opinions, it is believed that the medulloblastoma is only classified into four main subgroups, WNT, SHH, Group 3, and Group 4. There are also significant differences in the age distribution among different subgroups, and the Sonic Hedgehog subgroup (SHH subgroup) occurs with a typical bimodal distribution in different age groups. Specifically, the SHH subgroup often occurs in infants and adults, and about 59% of medulloblastoma in adult is SHH subgroup. Most of WNT subgroup occurs in children. The Group 3 subgroup commonly occurs in infants, while the Group 4 subgroup usually occurs in children and adults.

Identifying different subtypes is not only of great significance for understanding medulloblastoma, but also provides help for a clinical diagnosis and treatment. For example, a better prognosis is often obtained after a standard treatment is applied to W1NT subgroup; and, the use of some small molecular pathway inhibitors is very helpful for SHE subgroup medulloblastoma.

However, the current diagnosis of Sonic Hedgehog subgroup medulloblastoma (MBSHH) mainly refers to its gene expression profile obtained by RNA transcriptome sequencing, which has a high cost and a long-term period for experiments and analyses. As RNA is easily degraded and the technical requirements are also high for its experimental operations, the failure rate and error coefficient for the experiment are relatively high accordingly.

### SUMMARY

In order to overcome the above drawbacks, an aspect relates to a use of the methylated locus in Sonic Hedgehog subgroup medulloblastoma (MBSHH), and adopts the methylated locus in Sonic Hedgehog subgroup medulloblastoma into a diagnostic kit and a detection system. Using the diagnostic kit and system, only several methylated loci are targeted and detected, and Sonic Hedgehog subgroup medulloblastoma can be classified and identified. This use costs less than a tenth of that for transcriptome sequencing, and has a short experiment period. In addition, an object is subject to DNA and the experiment has a higher rate of success.

An aspect relates to use of a reagent for detecting methylation level of at least one genetic locus selected from ROBO4, OTX2OS1, AHRR, DKK4, and PART1 in a biological sample in the preparation of a diagnostic reagent or a diagnostic kit for Sonic Hedgehog subgroup medulloblastoma.

Based on analysis and comparison of data from a large number of MBSHH samples and other medulloblastoma subgroups samples (including other medulloblastoma subgroups, such as WNT, Group 3, and Group 4) a methylation level of genetic loci in the above genes can be used as a referenced indicator for diagnosis of MBSHH. Therefore, only a few methylated loci are targeted and detected, and MBSHH can be classified and identified, thereby reducing diagnostic difficulty and cost of MBSHH.

The biological sample may be a post-surgery tissue, or a tumor tissue obtained by other means.

It can be understood that said reagent may be prepared into a diagnostic kit, but also may be integrated into a detection equipment.

Another aspect relates to a diagnostic kit for detecting MBSHH, comprising a reagent for detecting a methylation level of at least one genetic locus selected from ROBO4, OTX2OS1, AHRR, DKK4, and PART1.

The diagnostic kit can be used for detecting a methylation level in at least one genetic locus selected from ROBO4, OTX2OS1, AHRR, DKK4, and PART1 in a biological sample, thus assisting the diagnosis of MBSHH or providing medication guidance for MBSHH.

It can be understood that the detection method can be conventional methods such as methylation microarray, methylation specific PCR, bisulfite sequencing, or other methods, which can detect the methylation level of the above loci.

In one example, a locus in ROBO4 gene comprises at least one of cg20419291, cg09684429, and cg19764370.

In one example, a locus in OTX2OS1 gene comprises at least one of cg22967396, cg23974194, cg04544856, cg14248715, and cg05732979.

In one example, a locus in AHRR gene comprises at least one of cg02385153, cg24064903, cg24980413, and cg16336872.

In one example, a locus in DKK4 gene comprises at least one of cg02571142, and cg09297903.

In one example, a locus in PART1 gene is cg26353176.

Another aspect relates to a detection system for MBSHH, comprising modules as follows: a detection module, for detecting a methylation level of the above loci by using the diagnostic kit hereinbefore; and an analysis module, for obtaining a result from the detection and comparing the result with the predetermined values of the methylation level of each locus, and finally getting a detection result of MBSHH.

The above detection system is used for classifying and identifying MBSHH by only targeting and detecting some methylated loci, thereby reducing diagnostic difficulty and cost of MBSHH.

In one example, the methylation level is methylation value (b-value), which refers to a methylation ratio of a locus. As a judgment standard for methylation level, b-value is simple and intuitive.

In one example, in the analysis module, when the b-value of cg20419291 is above 0.520, the b-value of cg09684429 is above 0.371, the b-value of cg19764370 is above 0.641, the b-value of cg22967396 is above 0.317, the b-value of cg23974194 is above 0.387, the b-value of cg04548856 is above 0.389, the b-value of cg14248715 is above 0.619, the b-value of cg05732979 is above 0.404, the b-value of cg02385153 is above 0.594, the b-value of cg24064903 is above 0.412, the b-value of cg24980413 is above 0.754, the b-value of cg16336872 is above 0.733, the b-value of cg02571142 is above 0.430, b-value of cg09297903 is above 0.440, and/or the b-value of cg26353176 is above 0.564, a sample may be MBSHH.

Based on screening and comparing a large number of samples, an optimal critical value for identifying MBSHH from other subgroups of medulloblastoma was obtained. Using this value as a judgement standard can provide a basis for the diagnosis of MBSHH.

Compared with the prior art, the present disclosure has the following benefits:

Based on analysis and comparison of data from a large number of MBSHH samples and OTHERS samples a use of a methylated locus of MBSHH of the present disclosure was found. The methylation level of a locus selected from ROBO4, OTX2OS1, AHRR, DKK4, and PART1 can be used as a referenced indicator for the diagnosis of MBSHH. Therefore, only a few methylated loci are targeted and detected, and MBSHH can be classified and identified, thereby reducing diagnostic difficulty and cost of MBSHH.

### BRIEF DESCRIPTION

Some of examples will be described in detail, with reference to the flowing figures, wherein like designations denote like members, wherein:

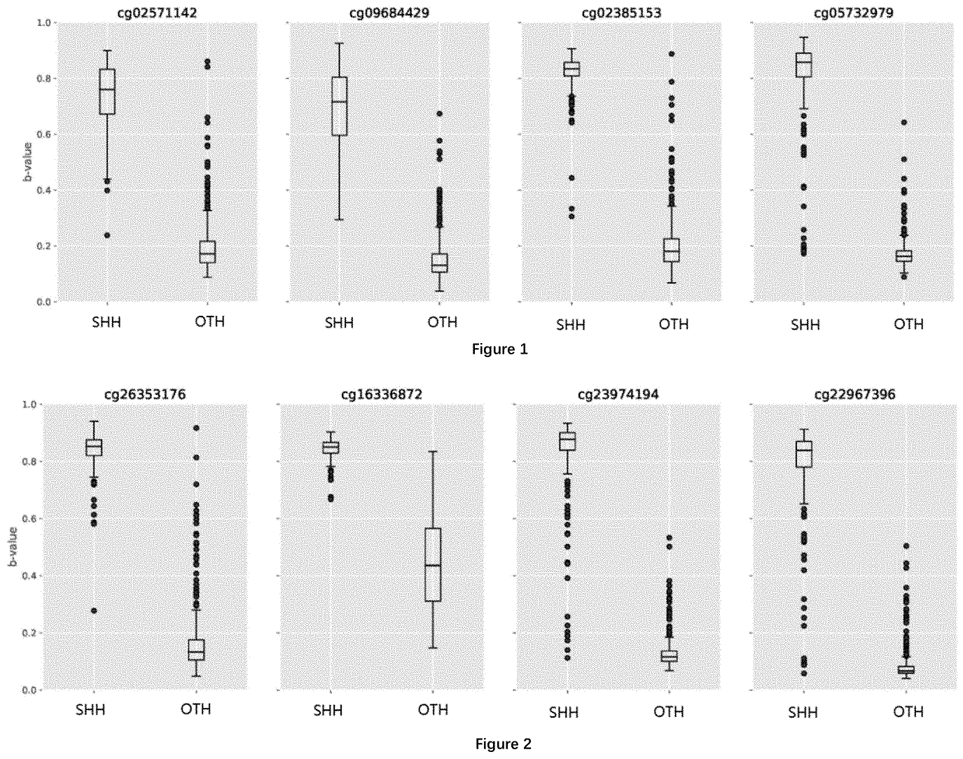

FIG. 1 depicts a box-plot showing b-values of the loci, cg26353176, cg16336872, cg23974194, and cg22967396 in Example 2; FIG. 2 depicts a box-plot showing b-values of the loci, cg02571142, cg09684429, cg02385153, and cg05732979 in Example 2;

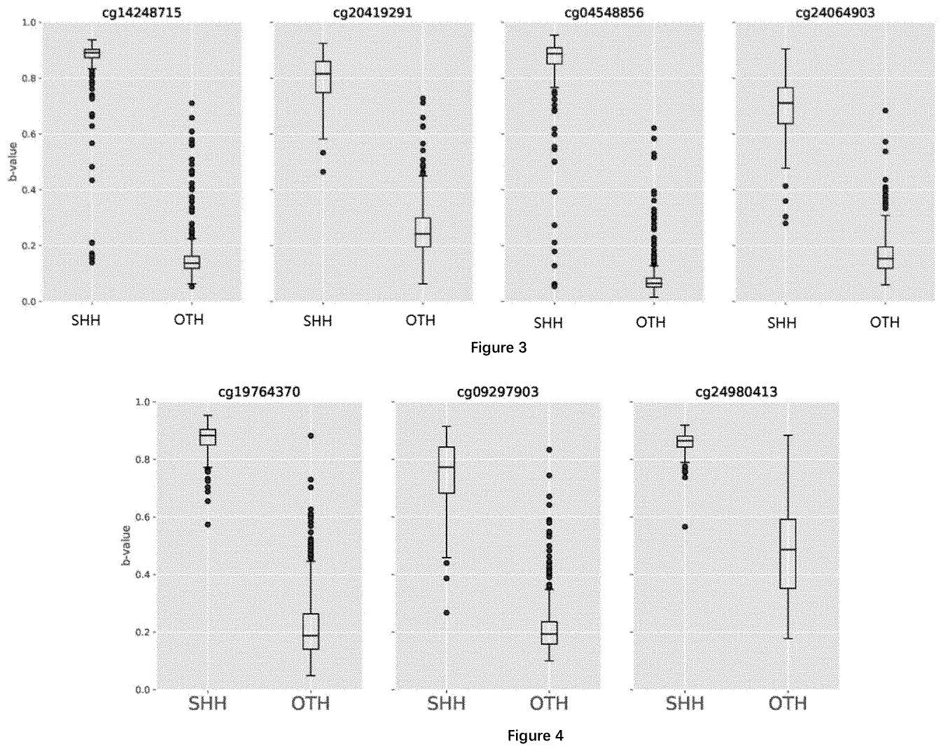

FIG. 3 depicts a box-plot showing b-values of the loci, cg14248715, cg20419291, cg04548856, and cg24064903 in Example 2;

FIG. 4 depicts a box-plot showing b-values of the loci, cg19764370, cg09297903, and cg24980413 in Example 2;

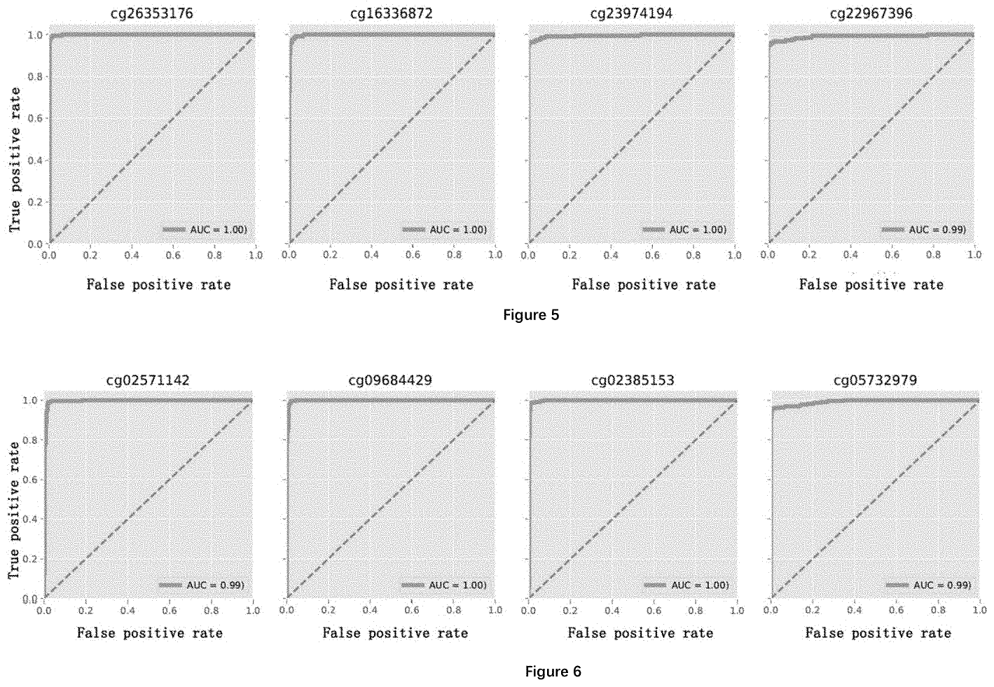

FIG. 5 depicts a receiver operating characteristic curve (ROC curve) of the loci, cg26353176, cg16336872, cg23974194, and cg22967396 in Example 2;

FIG. 6 depicts a ROC curve of the loci, cg02571142, cg09684429, cg02385153, and cg05732979 in Example 2;

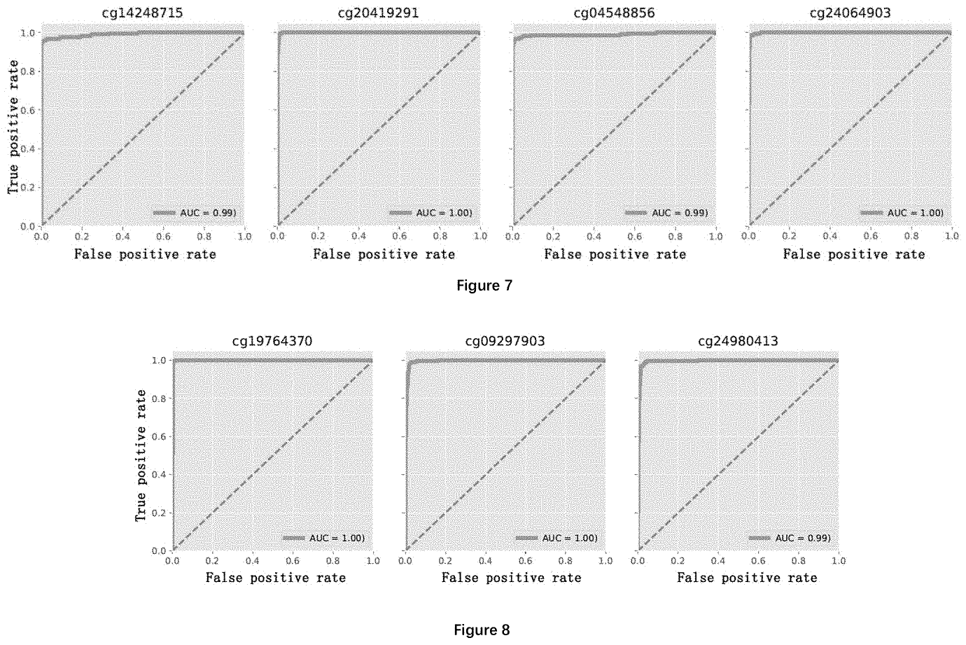

FIG. 7 depicts a ROC curve of the loci, cg14248715, cg20419291, cg04548856, and cg24064903 in Example 2;

FIG. 8 depicts a ROC curve of loci, cg19764370, cg09297903, and cg24980413 in Example 2; and

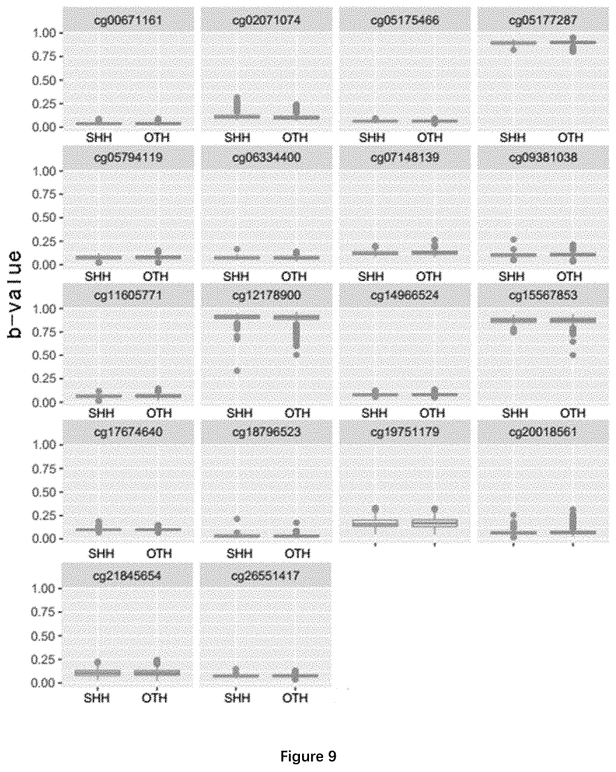

FIG. 9 depicts a box-plot showing a b-value of the methylated loci at ATP7B gene.

### DETAILED DESCRIPTION

For better understanding the present disclosure, the present disclosure will be fully illustrated hereinafter with reference to the accompanying figures. Preferred examples of the present disclosure are provided in the figures. However, the present disclosure may be realized in different forms, but is not limited to the examples descried herein. Instead, the purpose of providing the examples is to achieve a more thorough and comprehensive understanding of the present disclosure.

Unless otherwise defined, all technical and scientific terms used herein have the same meaning as those commonly understood by one skilled in the art in the technical field of the present disclosure. The terms used in the specification of the present disclosure are only to describe the specific examples and are not intended to limit the present disclosure. The term “and/or” used herein includes any and all combinations of one or more relevant listed items.

### Example 1

Collection and Detection of Samples

I. Collection of Samples

223 postoperative tissue samples were collected from patients with MB SHE, and 540 postoperative tissue samples were collected from patients with other medulloblastoma subgroups except MB SHE, that is, OTHERS samples (including other medulloblastoma subgroups, such as WNT, Group 3, and Group 4).

II. Detection of Samples

A commercially available Illumina Human Methylation 450 Bead Chip was used for detecting the methylation level of samples in a general experiment procedure. The experiment procedure includes the following steps:

1. extracting and amplifying DNA of the samples;

2. cleaving DNA into DNA fragments with enzymes;

3. isolating and purifying the DNA fragments, and then adding adaptor to them;

4. hybridizing the DNA fragments on microarray; and

5. detecting the microarray in a machine.

### Example 2

Screening of the Methylated Loci

I. Primary Screening of the Methylated Loci

A plurality of models was firstly built according to the data from Example 1, which served as a database. Each model was corresponding to a single methylated locus. Then the highly differential methylated loci were selected to construct a comprehensive model for a series of methylated loci, followed by obtaining an eigenvector of one sample from the b-value of the selected (one or more) methylated loci. And each sample was corresponding to one tumor subgroup (MB SHE or OTHERS), therefore a support vector machine (SVM) classification model was constructed to further obtain significantly differential methylated loci as shown in the following table.

TABLE 1
SIGNIFICANTLY DIFFERENTIAL METHYLATED
LOCI IN MBSHH AND OTHERS

| | | CHRO- | LOCUS | POSITIVE/ |
|---|---|---|---|---|
| | | MO- | COORDINATES IN | NEGATIVE |
| LOCI | GENES | SOMES | CHROMOSOMES | CHAIN |
| cg20419291 | ROBO4 | chr11 | 124767974 | + |
| cg09684429 | ROBO4 | chr11 | 124768015 | − |
| cg19764370 | ROBO4 | chr11 | 124767933 | + |
| cg22967396 | OTX2OS1 | chr14 | 57283942 | + |
| cg23974194 | OTX2OS1 | chr14 | 57284319 | − |
| cg04548856 | OTX2OS1 | chr14 | 57284528 | − |
| cg14248715 | OTX2OS1 | chr14 | 57284096 | − |
| cg05732979 | OTX2OS1 | chr14 | 57284219 | + |
| cg02385153 | AHRR | chr5 | 404766 | + |
| cg24064903 | AHRR | chr5 | 404910 | + |
| cg24980413 | AHRR | chr5 | 346987 | − |
| cg16336872 | AHRR | chr5 | 435267 | − |
| cg02571142 | DKK4 | chr8 | 42234803 | − |
| cg09297903 | DKK4 | chr8 | 42234798 | − |
| cg26353176 | PART1 | chr5 | 59783906 | − |

NOTE:
“+” represents a positive chain, and “−” represents a negative chain.

II. Validation of the Methylated Loci

1. Comparison for b-value.

The b-value of the above loci in patients with MBSHH and patients with OTHERS was analyzed and the results were shown in the following table and FIGS. 1-4.

TABLE 2
COMPARISON FOR B-VALUE OF MBSHH AND OTHERS

| | OTHERS (OTH) | | SHH | |
|---|---|---|---|---|
| | (540 CASES) | | (233 CASES) | |
| LOCI | AVERAGES | std | AVERAGES | std |
| cg26353176 | 0.1590 | 0.1023 | 0.8389 | 0.0647 |
| cg16336872 | 0.4405 | 0.1565 | 0.8443 | 0.0334 |
| cg23974194 | 0.1267 | 0.0489 | 0.8283 | 0.1524 |
| cg22967396 | 0.0801 | 0.0495 | 0.7794 | 0.1689 |
| cg02571142 | 0.1927 | 0.0872 | 0.7322 | 0.1233 |
| cg09684429 | 0.1500 | 0.0724 | 0.6892 | 0.1414 |
| cg02385153 | 0.1990 | 0.0922 | 0.8186 | 0.0740 |
| cg05732979 | 0.1714 | 0.0477 | 0.8086 | 0.1547 |
| cg14248715 | 0.1572 | 0.0842 | 0.8534 | 0.1379 |
| cg20419291 | 0.2565 | 0.0891 | 0.8008 | 0.0779 |
| cg04548856 | 0.0821 | 0.0662 | 0.8435 | 0.1501 |
| cg24064903 | 0.1662 | 0.0695 | 0.6988 | 0.1035 |
| cg19764370 | 0.2173 | 0.1146 | 0.8699 | 0.0546 |
| cg09297903 | 0.2131 | 0.0888 | 0.7491 | 0.1181 |
| cg24980413 | 0.4811 | 0.1488 | 0.8579 | 0.0375 |

FIGS. 1-4 are box-plots showing the b-value of different loci, wherein each middle line of each box in the figures represents an average value of the corresponding b-value.

It is known that methylation level of the above loci in MBSHH is higher than that in OTHERS according to the above results. Based on this, a high methylation level of the above loci can be used as a referenced indicator for the diagnosis of MBSHH.

2. Verification of ROC-AUC

A receiver operating characteristic curve (ROC curve) of the above loci in the subject was drawn. The results were shown in FIG. 5, wherein the abscissa represents a false positive rate, the ordinate represents a true positive rate, and the AUC value represents the area under the curve. The closer the AUC value for the ROC curve approaches to 1, the higher diagnostic value the indicator has.

It can be seen from the above results, that the ROC-AUC value of cg26353176 is 1.00, the ROC-AUC value of cg16336872 is 1.00, the ROC-AUC value of cg23974194 is 1.00, the ROC-AUC value of cg22967396 is 0.99, the ROC-AUC value of cg02571142 is 0.99, the ROC-AUC value of cg09684429 is 1.00, the ROC-AUC value of cg02385153 is 1.00, the ROC-AUC value of cg05732979 is 0.99, the ROC-AUC value of cg14248715 is 0.99, the ROC-AUC value of cg20419291 is 1.00, the ROC-AUC value of cg04548856 is 0.99, the ROC-AUC value of cg24064903 is 1.00, the ROC-AUC value of cg19764370 is 1.00, the ROC-AUC value of cg09297903 is 1.00, and the ROC-AUC value of cg24980413 is 0.99. Thus, all of the ROC-AUC values are above 0.99, which means that the methylation level of the above loci has a better capacity of identifying MBSHH.

3. Analysis of b-Value of Each Locus in MBSHH

3.1 Analysis of the b-Value of Each Locus

It can be seen from the results obtained from the above analysis of the b-value of the highly differential methylated locus in MBSHH, that the b-value in MBSHH is relatively highly centralized, thus MBSHH can be differentiated from the OTHERS, and the b-value in MBSHH can be used as an auxiliary judgement standard.

3.2 Analysis of a Critical Value of the b-Value of Each Locus

The critical value of b-value was calculated according to the comprehensive model, and the results were shown in the following table.

TABLE 3
CRITICAL VALUE OF THE B-VALUE

| | CRITICAL VALUE | | |
|---|---|---|---|
| LOCI | OF THE B-VALUE | FPR | TPR |
| cg26353176 | 0.564 | 0.017 | 0.996 |
| cg16336872 | 0.733 | 0.030 | 0.991 |
| cg23974194 | 0.387 | 0.004 | 0.964 |
| cg22967396 | 0.317 | 0.009 | 0.955 |
| cg02571142 | 0.430 | 0.020 | 0.991 |
| cg09684429 | 0.371 | 0.020 | 0.996 |
| cg02385153 | 0.594 | 0.011 | 0.987 |
| cg05732979 | 0.404 | 0.006 | 0.955 |
| cg14248715 | 0.619 | 0.004 | 0.955 |
| cg20419291 | 0.520 | 0.013 | 0.996 |
| cg04548856 | 0.389 | 0.009 | 0.969 |
| cg24064903 | 0.412 | 0.007 | 0.987 |
| cg19764370 | 0.641 | 0.006 | 0.996 |
| cg09297903 | 0.440 | 0.028 | 0.991 |
| cg24980413 | 0.754 | 0.035 | 0.991 |

FPR represents “false positive rate”; TPR represents “true positive rate”.

The FPR and TPR were shown in the above table when the above critical value of b-value was used as a boundary for classification. The selection of the critical value made the FPR and TPR reach an optimal balance, that is, the tangent point of a projection on the ROC curve. When such value is used as a judgment standard, it can provide a good basis for the diagnosis of MBSHH.

4. Verification of Control

ATP7B gene was selected at random and the methylation level of some loci in the ATP7B gene was detected and analyzed. The results were shown in FIG. 9, which showed the box-plots of b-value of each methylated locus in ATP7B gene in MB SHE and OTHERS, respectively. The results indicated that there was almost no difference in methylation level of the samples between the two groups.

The technical features of the above-mentioned embodiments can be combined arbitrarily. In order to make the description concise, not all possible combinations of the various technical features in the above-mentioned embodiments are described. However, all combinations of these technical features should be considered to fall within the scope of the specification as long as they have no contradiction.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of preparing a diagnostic reagent or a diagnostic kit for Sonic Hedgehog subgroup medulloblastoma comprising:
applying a reagent for detecting a methylation level of at least one genetic locus selected from ROBO4, OTX2OS1, AHRR, DKK4, and PART1 in a biological sample.

2. A diagnostic kit for detecting Sonic Hedgehog subgroup medulloblastoma, comprising a reagent for detecting a methylation level of at least one genetic locus selected from ROBO4, OTX2OS1, AHRR, DKK4, and PART1.

3. The diagnostic kit for detecting Sonic Hedgehog subgroup medulloblastoma according to claim 2, wherein the at least one genetic locus is in ROBO4 and comprises at least one of cg20419291, cg09684429, and cg19764370.

4. The diagnostic kit for detecting Sonic Hedgehog subgroup medulloblastoma according to claim 2, wherein the at least one genetic locus is in OTX2OS1 and comprises at least one of cg22967396, cg23974194, cg04544856, cg14248715, and cg05732979.

5. The diagnostic kit for detecting Sonic Hedgehog subgroup medulloblastoma according to claim 2, wherein the at least one genetic locus is in AHRR and comprises at least one of cg02385153, cg24064903, cg24980413, and cg16336872.

6. The diagnostic kit for detecting Sonic Hedgehog subgroup medulloblastoma according to claim 2, wherein the at least one genetic locus is in DKK4 and comprises at least one of cg02571142, and cg09297903.

7. The diagnostic kit for detecting Sonic Hedgehog subgroup medulloblastoma according to claim 2, wherein the at least one genetic locus is in PART1 and is cg26353176.

8. A detection system for Sonic Hedgehog subgroup medulloblastoma, comprising the following modules:
a detection module, for detecting a methylation level at least one genetic locus selected from ROBO4, OTX2OS1, AHRR, DKK4, and PART1; and
an analysis module, for obtaining a result from the detection and comparing the result with predetermined values of the methylation level at the respective at least one genetic locus, to get a detection result for identifying Sonic Hedgehog subgroup medulloblastoma.

9. The detection system for Sonic Hedgehog subgroup medulloblastoma according to claim 8, wherein the methylation level is b-value.

10. The detection system for Sonic Hedgehog subgroup medulloblastoma according to claim 9, wherein when the b-value of cg20419291 is above 0.520, the b-value of cg09684429 is above 0.371, the b-value of cg19764370 is above 0.641, the b-value of cg22967396 is above 0.317, the b-value of cg23974194 is above 0.387, the b-value of cg04548856 is above 0.389, the b-value of cg14248715 is above 0.619, the b-value of cg05732979 is above 0.404, the b-value of cg02385153 is above 0.594, the b-value of cg24064903 is above 0.412, the b-value of cg24980413 is above 0.754, the b-value of cg16336872 is above 0.733, the b-value of cg02571142 is above 0.430, b-value of cg09297903 is above 0.440, and/or the b-value of cg26353176 is above 0.564, a sample is Sonic Hedgehog subgroup medulloblastoma.
